(19) United States
(12) Patent Application Publication
(10) Pub. No.: US 2021/0138097 A1
(43) Pub. Date: May 13, 2021

(54) METHODS AND COMPOSITIONS FOR DRUG TARGETED DELIVERY

(71) Applicant: Jonathan M. Protz, Panama City (US)

(72) Inventor: Jonathan M. Protz, Panama City (US)

(21) Appl. No.: 17/053,491

(22) PCT Filed: May 8, 2019
(86) PCT No.: PCT/US2019/031395

§ 371 Date: Nov. 6, 2020

Related U.S. Application Data
(60) Provisional application No. 62/668,466, filed on May 8, 2018.
(60) Provisional application No. 62/682,036, filed on Jun. 7, 2018.
(60) Provisional application No. 62/763,362, filed on Jun. 12, 2018.
(60) Provisional application No. 62/764,313, filed on Jul. 27, 2018.
(60) Provisional application No. 62/764,551, filed on Aug. 8, 2018.
(60) Provisional application No. 62/765,833, filed on Sep. 15, 2018.
(60) Provisional application No. 62/765,956, filed on Sep. 21, 2018.
(60) Provisional application No. 62/766,019, filed on Sep. 25, 2018.

(51) Int. Cl. A61K 51/12; A61K 47/69

(52) U.S. Cl. CPC A61K 51/1255; A61K 47/6929

(57) ABSTRACT

Provided are methods for targeted drug delivery via mechanisms that use a particle's internal estimate of its own location within the body to target drug release at points specified on the basis of off-line medical imaging In some embodiments, the method relate to delivery that is accomplished by tailoring a material's composition so that it releases drugs or a chemical marker or dye when exposed to a specific sequence of environmental conditions or some set of specific sequences of environmental conditions, but does not do so when exposed to other such sequences.

### PRIORITY CLAIM

This application claims the priority benefit of U.S. Provisional Patent Application Ser. No. 62/682,036, filed Jun. 7, 2018 and U.S. Provisional Patent Application Ser. No. 62/668,466 filed on May 8, 2018, the disclosures of which are incorporated herein by reference in their entireties.

### TECHNICAL FIELD

The presently disclosed subject matter relates primarily to the technology of pharmaceuticals and their delivery in animals and to chemicals and their delivery in plants, but can also find application in any scenario requiring the location-specific release or application of chemicals or other atoms, molecules, or small particles. Examples of the latter might include the selective patterning of surfaces in the processing or manufacture of semiconductors, electronic devices, and other micro devices.

### BACKGROUND

Targeted drug delivery is an area of active investigation and has been for several decades. Most approaches target chemically via cell-borne receptors or via genetics. Some use ex vivo stimulus such as heat or radio waves to drive spatially-localized release.

There are a variety of approaches to increase the specificity of the delivery or activity of a drug. Some approaches target delivery or activity on the basis of chemical interactions with chemical markers, receptors, or other such signatures exhibited by target cells. Others target delivery or activity with the assistance of an external aid that provides a signal for release. For example, a drug delivery vehicle can be sensitive to temperature and can release in localities where an external heat pack is applied. Or, a drug delivery vehicle can be sensitive to radio-frequency radiation and can release in the proximity of a radiating antennae.

Approaches of the first type, which target chemical markers or other similar signatures, have the drawback of requiring some knowledge about the disease or cell type being targeted and also of being category-selective rather than location-selective. Approaches of the second type, which are activated or released by proximity to or in response to an external aid, are location-selective, but require the use of both the drug particle itself and the external aid.

The co-inventor's previous work in this area (see e.g., U.S. Patent Application Publication No. 2009/0275031; incorporated herein in its entirety) focused on the development of nanoparticles capable of sensing and retaining a memory of their environment. Droplets of suspensions of DNA-charged liposomes and enzymes stitched within themselves DNA chains that noisily recorded the temperature history experienced by the droplets.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list all possible combinations of such features.

In some embodiments, the presently disclosed subject matter provides methods for targeted drug delivery. In some embodiments, the methods employ particles that comprise one or more drugs, wherein the particles' internal estimates of their locations within a subject's body are employed to target drug release of the one or more drugs at points specified on the basis of off-line medical imaging. In some embodiments, the particle's estimate is formed in part on the basis of information it has detected and recorded about the environment of its recent past.

In some embodiments, delivery is accomplished by tailoring a material's composition so that it releases drugs or a chemical marker or dye when exposed to a specific sequence of environmental conditions or some set of specific sequences of environmental conditions, but does not do so when exposed to other such sequences. In some embodiments, the specific environmental sequence corresponds to the particle being at a specific location, such as at a unique cluster of capillaries, within the body of an animal or the vascular system of a plant or within some other branched network.

In some embodiments, delivery is accomplished by tailoring a synthetic biological organism or mechanism so that it expresses a specific gene or releases a drug or a chemical when exposed to a specific sequence of environmental conditions or some set of specific sequences of environmental conditions, but does not do so when exposed to other such sequences. In some embodiments, the particle's estimate is formed in part on the basis of information it has detected and recorded about the environment of its recent past. In some embodiments, the specific environmental sequence corresponds to the particle being at a specific location, such as at a unique cluster of capillaries, within the body of an animal or the vascular system of a plant or within some other branched network such as a network of pipes.

In some embodiments, the passage of a tailored material or synthetic biological organism or system through some network of vessels, pipes, channels, or other contained and interconnected volumes is treated as a brute force decryption, with the targeted capillary as message, the location-sensitive particle as cryptogram, the trailing history of branch environments as trial key, circulation as a random cycling of trial keys, release as a successful decryption, and particle design as a problem of robust optimization of material parameters with a goal of balancing type I and II errors in release.

In some embodiments, the particle includes an endowment of usable energy or a mechanism for energy harvesting and storage and wherein it is able to use the endowed or harvested and stored energy to influence its movement through the circulatory system in such a way as to increase the frequency or likelihood of it visiting a desired target location.

In some embodiments, the presently disclosed subject matter also provides methods for drug delivery, wherein the probability of a circulating drug particle taking one branch over another at one or more junctions in the circulatory system is controlled by a mechanism that couples changes in some expressed feature or collection of expressed features of the particle that affects its interaction with its environment to the environmental conditions at or leading up to the branch in such a manner as to increase the likelihood of the particle visiting or revisiting a particular targeted area through its course of circulation. In some embodiments, the expressed feature of the particle is its buoyancy. In some embodiments, the expressed feature of the particle is its coefficient of drag. In some embodiments, the expressed feature of the particle is its electric charge. In some embodiments, the expressed feature of the particle is a combination of its buoyancy and its coefficient of drag. In some embodiments, the mechanism which couples the environmental conditions to changes in the expressed feature is a tailored material. In some embodiments, the mechanism which couples the environmental conditions to changes in the expressed feature is a tailored composite material. In some embodiments, the tailored material is designed by solving an optimization problem that specifies the composition of the material which maximizes time spent at the target site while minimizing or constraining the amount of time spent at any other particular site. In some embodiments, the tailored material is designed by solving an optimization problem that specifies the composition of the material which maximizes the delivery rate in the vicinity of the target site while minimizing or constraining the delivery rate at any other particular site. In some embodiments, the mechanism which couples the environmental conditions to changes in the expressed feature is a synthetic biological mechanism. In some embodiments, the mechanism which couples the environmental conditions to changes in the expressed feature involves a recording in DNA of the recent history of environmental conditions experienced by the particle or other delivery vehicle. In some embodiments, the synthetic biological system is designed by solving an optimization problem that maximizes time spent at the target site or release rate in the vicinity of the target site while minimizing or constraining the amount of time spent at any other particular site or the release rate at these other sites. In some embodiments, the permeability or porosity or some other internal feature of the delivery particle which controls rate of release is also regulated and the design of the particle for steering and release is coupled. In some embodiments, the regulatory mechanism is designed by solving simultaneous optimization problems for both steering and release in such a manner that release rate is maximized in the vicinity of the target site and minimized or constrained elsewhere. In some embodiments, a formulation comprising potentially several components is employed rather than a specific particle, and the formulation collectively implements the regulation mechanism. In some embodiments. the formulation is composed of a steered particle that carries and releases a drug and is sensitive to the concentration of two markers, a release marker and a steering marker, a particle that carries and releases a steering marker, and a particle that releases a release marker. In some embodiments, the formulation is composed of a steered particle that carries and releases a drug and is sensitive to the concentration of two markers, a release marker and a steering marker, a particle that carries and releases both of these markers.

Thus, in some embodiments the presently disclosed subject matter relates to methods for targeted drug delivery via a mechanism that uses a particle's internal estimate of its own location within a subject's body to target release of a drug contained therein and/or thereon at a point specified by offline medical imaging. In some embodiments, the particle's estimate is formed in part on the basis of information it detects and records about the environment of its recent past. In some embodiments, delivery is accomplished by tailoring the particle's composition so that it releases the drug when exposed to a specific sequence of environmental conditions or some set of specific sequences of environmental conditions, but does not do so when exposed to the specific sequence of environmental conditions or the set of specific sequences of environmental conditions. In some embodiments, the specific sequence of environmental conditions corresponds to the particle being at a specific location, such as at a unique cluster of capillaries, within the body of an animal or the vascular system of a plant. In some embodiments, the particle comprises a synthetic biological organism or mechanism and delivery is accomplished by tailoring the synthetic biological organism or mechanism so that it expresses a specific gene or releases a drug or a chemical when exposed to a specific sequence of environmental conditions or some set of specific sequences of environmental conditions, but does not do so when exposed to the specific sequence of environmental conditions or the set of specific sequences of environmental conditions. In some embodiments, wherein the particle's estimate is formed in part on the basis of information it detects and records about the environment of its recent past. In some embodiments, the specific sequence of environmental conditions corresponds to the particle being at a specific location, such as at a unique cluster of capillaries, within the body of an animal or the vascular system of a plant. In some embodiments, passage of a tailored material or synthetic biological organism or system through some network of vessels can be treated as a brute force decryption, with the targeted capillary as message, the location-sensitive particle as cryptogram, the trailing history of branch environments as trial key, circulation as a random cycling of trial keys, release as a successful decryption, and particle design as a problem of robust optimization of material parameters with a goal of balancing type I and II errors in release.

In some embodiments, the particle comprises an endowment of usable energy or a mechanism for energy harvesting and storage, and further wherein the particle is constructed to use the endowed or harvested and stored energy to influence its movement through the circulatory system in such a way as to increase the frequency or likelihood of it visiting a desired target location.

The presently disclosed subject matter also relates in some embodiments to methods for drug delivery wherein the probability of a circulating drug particle taking one branch over another at one or more junctions in a subject's circulatory system is controlled by a mechanism that couples changes in some expressed feature or collection of expressed features of the particle that affects its interaction with its environment to the environmental conditions at or leading up to the branch in such a manner as to increase the likelihood of the particle visiting or revisiting a particular targeted area through its course of circulation. In some embodiments, the expressed feature of the particle is its buoyancy, its coefficient of drag, its electric charge, or any combination thereof. In some embodiments, the expressed feature or collection of expressed features of the particle is a combination of its buoyancy and its coefficient of drag. In some embodiments, the mechanism which couples the environmental conditions to changes in the expressed feature is a tailored material. In some embodiments, the mechanism that couples the environmental conditions to changes in the expressed feature is a tailored composite material. In some embodiments, the tailored composite material is designed by solving an optimization problem that specifies the composition of the material which maximizes time spent at the target site while minimizing or constraining the amount of time spent at any other particular site. In some embodiments, the tailored composite material is designed by solving an optimization problem that specifies the composition of the material which maximizes its rate of delivery to a target site while minimizing or constraining its rate of delivery to any or all non-target sites. In some embodiments, the mechanism that couples the environmental conditions to changes in the expressed feature is a synthetic biological mechanism. In some embodiments, the mechanism that couples the environmental conditions to changes in the expressed feature involves a recording in DNA of the recent history of environmental conditions experienced by the particle or other delivery vehicle. In some embodiments, the synthetic biological system is designed by solving an optimization problem that maximizes time spent at the target site and/or a release rate in the vicinity of the target site while minimizing or constraining the amount of time spent at any other particular site or the release rate at any other pre-determined site. In some embodiments, permeability, porosity, or some other internal feature of the particle that controls the rate of release of the drug is regulated and design of the particle for steering and release is coupled. In some embodiments, a regulatory mechanism is designed by solving simultaneous optimization problems for both steering and release in such a manner that release rate is maximized in the vicinity of the target site and minimized or constrained at any other pre-determined site. In some embodiments, a formulation comprising several components is employed rather than a singular particle, and the formulation collectively implements a regulation mechanism. In some embodiments, the formulation comprises a steered particle that carries and releases a drug and is sensitive to concentrations of a plurality of markers selected from the group consisting of a release marker, a steering marker, a particle that carries and releases a steering marker, and a particle that releases a release marker. In some embodiments, the formulation comprises a steered particle that carries and releases a drug and is sensitive to the concentration of two markers, a release marker and a steering marker, a particle that carries and releases both of these markers.

In some embodiments, the presently disclosed subject matter also relates to methods for fabricating environmentally-sensitive particles. In some embodiments, the methods comprise cladding a hydrogel or other hydrophilic medium with a comparatively impermeable layer, the latter of which has a permeability that is sensitive to environment, and thereafter arranging layers of such materials upon one another.

In some embodiments, the presently disclosed subject matter also relates to methods for fabricating impermeable films that are selective for their environments. In some embodiments, the methods comprise quilting together a plurality of tiles or bits of different materials, each with its own response to an environment to which the impermeable film might be exposed.

In some embodiments, the presently disclosed subject matter also relates to methods for fabricating impermeable films that are selective for its environment comprising depositing randomly in a lipid bilayer or other similar film a plurality of compounds that change at least one characteristic in response to different environmental stimuli.

In some embodiments, the presently disclosed subject matter also relates to methods for fabricating materials the permeability of which are sensitive to environmental stimuli comprising arranging layers of comparatively permeable material separated by layers of semipermeable material in such a way that the total path traveled by a diffusing particle depends on the distance separating pores in the semipermeable material, wherein the distance separating pores in the semipermeable material varies with different environmental stimuli.

In some embodiments, the presently disclosed subject matter also relates to uses of physical parameters, optionally material parameters, and geometry from a synthetic tissue model or whole-body synthetic tissue model to specify design parameters of a selective-release drug delivery mechanism.

In some embodiments, the presently disclosed subject matter also relates to uses of environmentally-sensitive materials, particles, and/or formulations to subject targeted release of a drug to a form of permissive action link.

In some embodiments, the presently disclosed subject matter also relates to methods for fabricating an environmentally-sensitive large pseudomolecule comprising extruding, patterning, or otherwise processing a strand of polymer that is locally doped, coated, or otherwise treated, thereby causing the strand to fold into a conformation that is sensitive in a pre-determined way to environmental stimuli. In some embodiments, the environmentally-sensitive large pseudomolecule is sensitive to environmental stimuli in a manner that results in a conformation of the environmentally-sensitive large pseudomolecule to vary different locations of a subject's a circulatory system.

In some embodiments, the presently disclosed subject matter also relates to methods for fabricating environmentally-sensitive large molecules comprising synthesizing the environmentally-sensitive large pseudomolecule from a sequence of monomers, the sequence of which results in the environmentally-sensitive large pseudomolecule to adopt different conformations in response to local environmental stimuli or to a particular sequence of local environmental stimuli in a pre-determined manner. In some embodiments, the environmentally-sensitive large pseudomolecule comprises a peptide, a protein, a protein complex, or a combination thereof, optionally wherein the monomers are amino acids.

In some embodiments, the presently disclosed subject matter also relates to methods for fabricating environmentally-sensitive large molecules comprising synthesizing the environmentally-sensitive large pseudomolecule from a sequence of monomers, the sequence of which results in the environmentally-sensitive large pseudomolecule adopting different conformations in response to different environmental stimuli at one or more locations within a subject's body, wherein at least one of the different conformations results in the environmentally-sensitive large pseudomolecule being therapeutically active and at least one of the different conformations results in the environmentally-sensitive large pseudomolecule being therapeutically inactive. In some embodiments, the environmentally-sensitive large pseudomolecule comprises a peptide, a protein, a protein complex, or a combination thereof, and optionally wherein the monomers are amino acids.

In some embodiments, the presently disclosed subject matter also relates to uses of physical parameters, optionally material parameters, and geometry from a synthetic tissue model or whole-body synthetic tissue model to specify the design parameters of large molecule drug or large molecule drug delivery composition, wherein the design parameters result in the large molecule drug or large molecule drug delivery composition selectively expressing its therapeutic activity or selectively concealing its therapeutic activity.

In some embodiments, the presently disclosed subject matter also relates to uses of environmentally-sensitive large molecules, tailored materials, particles, and/or other formulations to subject activity of a drug associated therewith to a permissive action link.

In some embodiments, the presently disclosed subject matter also relates to uses of environmentally-sensitive large molecules, materials, particles, and/or other formulations to subject release of a drug associated therewith to a permissive action link.

In some embodiments, the presently disclosed subject matter also relates to uses of environmentally-sensitive large molecules, tailored materials, particles, and/or other formulations to subject an activity of a drug associated therewith to targeting dependent on its location within a subject's body.

In some embodiments, the presently disclosed subject matter also relates to uses of environmentally-sensitive large molecules, tailored materials, particles, and/or other formulations to subject release of a drug associated therewith to targeting dependent on its location within a subject's body.

In some embodiments, the presently disclosed subject matter also relates to methods for synthesizing compositions, the conformations of which are sensitive to the compositions' locations within a subject's circulatory system. In some embodiments, the methods comprise selecting a sequence of monomers or another feature of the composition, such that the potential energy well of the composition comprises local minima and activation energy barriers that vary with environmental stimuli in such a way as to cause the composition to adopt a desired conformation at one or more pre-determined locations with a subject's body and one or more different pre-determined conformation at other locations within the subject's body. In some embodiments, the composition comprises a peptide, a protein, a protein complex, a polymer strand, or any combination thereof. In some embodiments, the composition comprises or is otherwise associated with a drug. In some embodiments, sensitivity of a shape of the potential energy well to the environmental stimuli is such that the composition adopts a specific conformation with high probability only when the particle traverses a pre-determined specific path or path segment within the subject's body. In some embodiments, the potential energy well comprises there three local minima, as a function of conformation, and three activation energy barriers separating them, with the barrier between the first and third always lowered for regions of a flow field on the return circuit, optionally the subject's veins, after a target location or non-target location, optionally the subject's capillaries, has been passed, with the first minimum the global minimum during that time, and with the barrier raised at all other times; where the barrier between the first and second is only lowered on branches leading to the target area, but not on branches that do not lead to the target area, so that the conformation can only change to the second conformation if the particle flows along a branch leading to the target area; and where the barrier between the second and third conformations is only lowered in the vicinity of a target or non-target (e.g., capillaries), but not during branches leading to or away from these areas, and with the barrier between the first and second kept raised, so that the third conformation can only be reached if the particle takes the correct path, which left it in the second conformation, it being left in the first conformation, even with the barrier between the second and third lowered, otherwise, due to the barrier between the first and second minima.

Thus, it is an object of the presently disclosed subject matter to provide methods for targeted drug delivery that do not depend on recognition of cell-borne receptors and instead result from particular characteristics of the microenvironment in which a drug carrier finds itself and/or a particular sequence of different microenvironments that the drug carrier experiences.

An object of the presently disclosed subject matter having been stated above, other objects and advantages will become apparent upon a review of the following Detailed Description.

### DETAILED DESCRIPTION

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a cell” refers to one or more cells, including a plurality of the cells. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

The term “subject” as used herein refers to a member of any invertebrate or vertebrate species. Accordingly, the term “subject” is intended to encompass any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals)), and all Orders and Families encompassed therein.

The compositions and methods of the presently disclosed subject matter are particularly useful for warm-blooded vertebrates. Thus, the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A goal of the presently disclosed subject matter is to allow the delivery of drugs to specific points within the body, such as to the vicinity of diseased tissue, without requiring the use or knowledge of chemical or biological markers uniquely associated with the targeted tissue and without requiring the involvement of external apparatus such as heating elements or radio transmitters. A core feature of the presently disclosed subject matter is one of targeting delivery using a particle's own estimates of particle location within the body to target drug release at points specified on the basis of off-line medical imaging. is closely related to the terrain correlation mapping (TERCOM) techniques used in aircraft navigation.

In some embodiments, particles, delivery vehicles, and formulations of the presently disclosed subject matter can estimate their own location within the body by correlating vectors of sensed environmental variables (e.g., temperature, pressure, salinity, sugar levels, pH, etc.) against a map release drugs or other chemicals at targeted sites on the basis of this location estimate. This approach eliminates the reliance upon ex vivo navigation aids and cell markers. This specificity of location is accomplished by tailoring material properties of an inanimate material particle or the genetics of a synthetic biological life form.

Thus, in some embodiments the particles, delivery vehicles, or formulations are sensitive to environmental stimuli in the various different environments within a subject's body, particularly within different systems of the subject's body as well as different locations within any given system (including but not limited to the circulatory system). Exemplary environmental stimuli can include, but are not limited to variations in temperature, pressure, salinity, sugar levels, pH, etc. As such, in some embodiments a particle, delivery vehicle, or formulation of the presently disclosed subject matter comprises a structure such as but not limited to a liposome or nanoparticle that alters its composition and/or conformation in response to variations in temperature, pressure, salinity, sugar levels, pH, etc. that it experiences as it traverses the subject's circulatory system. Exemplary delivery vehicles that can alter their compositions and/or conformations in response to variations in vivo environmental stimuli include, but are not limited to those disclosed in U.S. Pat. Nos. 7,780,979 (temperature-sensitive hydrogels), the entire disclosure of which is incorporated by reference.

Furthermore, polymers can be formed into gels by dispersing them into a solvent such as water. In certain embodiments, polysaccharides and polypeptides and other polymers can be fashioned to release microparticles and/or a therapeutic agent present in the microparticles upon exposure to a specific triggering event such as pH (see e.g., Heller et al. (1988) Chemically Self-Regulated Drug Delivery Systems. in Polymers in Medicine III, Elsevier Science Publishers B.V., Amsterdam, pp. 175-188; Peppas (1993) Fundamentals of pH- and Temperature-Sensitive Delivery Systems. in Gurny et al. (eds.), Pulsatile Drug Delivery, Wissenschaftliche Verlagsgesellschaft mbH, Stuttgart, pp. 41-55; Doelker (1993) Cellulose Derivatives. in Peppas and Langer (eds.), Biopolymers I, Springer-Verlag, Berlin). Representative examples of pH-sensitive polysaccharides include carboxymethyl cellulose, cellulose acetate trimellilate, hydroxypropylmethylcellulose phthalate, hydroxypropyl-methylcellulose acetate succinate, chitosan and alginates.

Similarly, polysaccharides and polypeptides and other polymers can be fashioned to be temperature sensitive (see e.g., Okano (1995) Molecular Design of Stimuli-Responsive Hydrogels for Temporal Controlled Drug Delivery. Proceed. Intern. Symp. Control. Rel. Bioact Mater. 22:111-112, Controlled Release Society, Inc.; Hoffman et al. (1993) Characterizing Pore Sizes and Water ‘Structure’ in Stimuli-Responsive Hydrogels. Center for Bioengineering, Univ. of Washington, Seattle, Wash., p. 828; Hoffman (1988) Thermally Reversible Hydrogels Containing Biologically Active Species. in Migliaresi et al. (eds.), Polymers in Medicine III, Elsevier Science Publishers B.V., Amsterdam, pp. 161-167; Hoffman (1987) Applications of Thermally Reversible Polymers and Hydrogels in

Therapeutics and Diagnostics. in Third International Symposium on Recent Advances in Drug Delivery Systems, Salt Lake City, Utah, Feb. 24-27, 1987, pp. 297-305). Representative examples of thermogelling polymers, such as poly(oxyethylene)-poly(oxypropylene) block copolymers (e.g., PLURONIC F127 from BASF Corporation, Mount Olive, N.J.), and cellulose derivatives. Paclitaxel microspheres having lower, traditional loadings have been incorporated into a thermoreversible gel carrier (PCT International Patent Application Publication No. WO 2000/066085).

Exemplary polysaccharides include, without limitation, hyaluronic acid (HA), also known as hyaluronan, and derivatives thereof (see e.g., U.S. Pat. Nos. 5,399,351; 5,266,563; 5,246,698; 5,143,724; 5,128,326; 5,099,013; 4,913,743; and 4,713,448), including esters, partial esters and salts of hyaluronic acid. For example, an aqueous solution of HA having a non-inflammatory molecular weight (greater than about 900 kDa) and a concentration of about 10 mg/ml would be in the form of a gel. The aqueous solution may further comprise one or more excipients that serve other functions, such as buffering, anti-microbial stabilization, or prevention of oxidation. Microspheres made from, for example, 70% paclitaxel loaded poly(L-lactide), MW=2000, may be incorporated into a 10 mg/ml HA gel as follows. HA, MW=1 MDa, is dissolved in water to a concentration of 20 mg/ml and microparticles are dispersed in water to a concentration in the range of 0.02 to 20 mg/ml. The two phases are combined in equal volumes by mixing (e.g., syringe mixing, using two interconnected luer lok syringes between which the liquids are passed back and forth fifty times), such that the microparticles are evenly distributed throughout the mixture, which has a concentration of 10 mg/ml HA and between 0.1 and 10 mg/ml microparticles, equivalent to 0.07 and 7 mg/ml paclitaxel in a gel carrier.

Also, other polymeric carriers can be fashioned which are temperature sensitive are known. See e.g., Chen et al. (1995) Novel Hydrogels of a Temperature-Sensitive Pluronic Grafted to a Bioadhesive Polyacrylic Acid Backbone for Vaginal Drug Delivery. Proceed. Intern. Symp. Control. Rel. Bioact. Mater. 22:167-168, Controlled Release Society, Inc.; Johnston et al. (1992) Pharm. Res. 9(3):425-433; Tung (1994) Intl J. Pharm. 107:85-90; Harsh & Gehrke (1991) J. Controlled Release 17:175-186; Bae et al. (1991) Pharm. Res. 8(4):531-537; Dinarvand & D'Emanuele (1995) J. Controlled Release 36:221-227; Yu & Grainger (1993) Novel Thermos-sensitive Amphiphilic Gels: Poly N-isopropylacrylamide-co-sodium acrylate-co-n-N-alkylacrylamide Network Synthesis and Physicochemical Characterization. Dept. of Chemical & Biological Sci., Oregon Graduate Institute of Science & Technology, Beaverton, Oreg., pp. 820-821; Zhou & Smid (1993) Physical Hydrogels of Associative Star Polymers. Polymer Research Institute, Dept. of Chemistry, College of Environmental Science and Forestry, State Univ. of New York, Syracuse, N.Y., pp. 822-823; Yu & Grainger (1993) Thermo-sensitive Swelling Behavior in Crosslinked N-isopropylacrylamide Networks: Cationic, Anionic and Ampholytic Hydrogels. Dept. of Chemical & Biological Sci., Oregon Graduate Institute of Science & Technology, Beaverton, Oreg., pp. 829-830; Kim et al. (1992) Pharm. Res. 9(3):283-290; Bae et al. (1991) Pharm. Res. 8(5):624-628; Kono et al. (1994) J. Controlled Release 30:69-75; Yoshida et al. (1994) J. Controlled Release 32:97-102; Okano et al. (1995) J. Controlled Release 36:125-133; Chun & Kim (1996) J. Controlled Release 38:39- 47; D'Emanuele & Dinarvand (1995) Intl J. Pharm. 118:237-242; Katono et al. (1991) J. Controlled Release 16:215-228; Gutowska et al. (1992) J. Controlled Release 22:95-104; Palasis & Gehrke (1992) J. Controlled Release 18:1-12; Paavola et al. (1995) Pharm. Res. 12(12):1997-2002.

Representative examples of thermogelling polymers, and their gelatin temperature (LCST; ° C.) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N,n-diethylacrylamide), 32.0; poly(N-isopropylmethacrylamide), 44.0; poly (N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide). Other representative examples of thermogelling polymers include cellulose ether derivatives such as hydroxypropyl cellulose, 41° C.; methyl cellulose, 55° C.; hydroxypropylmethyl cellulose, ° C.; and ethylhydroxyethyl cellulose, and Pluronics such as F-127, 10-15° C.; L-122, 19° C.; L-92, 26° C.; L-81, 20° C.; and L-61, 24° C.

In some embodiments, composite shells of polymer or lipid bilayer penetrated with a chowder of conformation-changing particles, each sensitive to different stimuli and with different thresholds, are tailored to change porosity after exposure to a path-specific sequence of environmental shifts.

In some embodiments, encapsulated droplets of environmentally-responsive suspensions might be engineered to release indicators whenever a specific environmental sequence is experienced.

Other embodiments are also envisioned. All share the features that they are composed of mixtures of materials with different sensitivities to environment and that one or more of the parameters of these different materials is selected in such a way as to ensure that when the composition experiences a particular sequence of shifts in its environment it responds in a specific, predictable manner, and when it does not, it does not.

Compositions can include within them polymer vessels, proteins, computational DNA, engineered cells, biodegradable nanoparticles, etc., can substitute; all are common in drug delivery research and have been explored by other investigators in a variety of comparable applications.

An advantage of the presently disclosed subject matter when applied to the medical applications is that it allows locally-selective drug delivery without requiring prior knowledge of disease markers or the genetics of the patient or disease, allowing the use instead of information gathered through medical imaging.

Also, the approaches disclosed herein, being stochastic in nature, can allow the development of libraries of delivery particles that are each selective for specific locations within the body, based on environmental variables, since each particle effectively carries its own internal map.

This filing focuses on targeted drug delivery mechanisms wherein internal estimates of particle location within a body or other interconnected network of volumes is used to target release of drugs or chemicals or the expression of genes or other such signatures at points specified on the basis of off-line medical imaging.

In some embodiments, the subject matter disclosed herein relates to mechanisms for estimating location within the body from vectors of sensed environmental variables (e.g., temperature, pressure, salinity, sugar levels, pH, etc.) or their trailing averages. Particularly, in some embodiments it uses particles or formulations that estimate their own location within the body by correlating such vectors of sensed environmental variables (e.g., temp., pressure, salinity, sugar levels, pH, etc.) against a map and release their drug at targeted sites on the basis of this location estimate.

Notionally, shells of polymer or lipid bilayer penetrated with a chowder of conformation-changing particles, each sensitive to different stimuli and with different thresholds, might be tailored to change porosity after exposure to a path-specific sequence of environmental shifts; or, encapsulated droplets of environmentally-responsive suspensions might be engineered to release indicators whenever a specific environmental sequence is experienced.

Registration of environmental terrain with anatomical location can be accomplished off-line through a multi-factor regression of blood samples, etc. drawn from a set of representative individuals at consistent locations throughout the body and under various contexts (e.g., wake. sleep, etc.), all collapsed onto a single non-dimensional parametric model representing the generalized morphology of the body or circulatory system. Traverse of the body can be modeled by a stochastic, discrete-state, continuous-transition Markov process with vascular branch or region as state.

Delivery can be modeled as brute force decryption, with the targeted capillary as message, the location-sensitive particle as cryptogram, the trailing history of branch environments as trial key, vascular circulation as a random cycling of trial keys, and release as a successful decryption.

Particle design can then be cast as a problem of robust optimization of material parameters with a goal of balancing type I and II errors in release.

Targeted drug delivery is an area of active investigation and has been for several decades. Most approaches target chemically via cell-borne receptors or via genetics. Some use ex vivo stimulus such as heat or radio waves to drive spatially-localized release. In the first case, drug-laden vessels formed from lipid bilayers (liposomes) with the special property that they become more porous when exposed to a temperature above some threshold. By using an external heat pack or heating element to apply heat or cold locally to some specific area of the body, these can be stimulated to release their drugs in this local region. In the second case, DNA strands tagged with gold atoms can be caused to open when exposed to radio waves of specific frequency, making the exposed regions available for transcription. By focusing radio waves from an external source on some specific region of the body, these can be cause to be available for transcription in a therapeutic capacity at that location.

There are many other potential embodiments like these. In some embodiments, they all have in common the feature that they rely on some means of stimulating drug release that is outside of the normal operation of the human body As set forth herein, the drug delivery particles are designed specifically to require the application of no external or unnatural stimulus and to be highly selective for a specific locale within the body.

A particular mathematical problem of interest is for the case of a continuous-transition, discrete-state Markov chain of with a single chain of finite extent and no periodic states; this is ergodic with stable long-run probabilities (if the chain is very large or infinite, it can be divided into a set including the initial state within which there is a high probability or remaining over some arbitrary time horizon, and a set which there is a low probability of entering). Such a chain is representative of the movement of blood and the particles it carries through the circulatory system. At each juncture, there is some probability of taking one branch over the other, but the system is closed so that every juncture can eventually be visited after a long enough period of time. A particle or system can move through this network with the particle or system having some structure that is capable of causing changes in its internal state (e.g., its porosity, whether a sensing mechanism is on, etc.) or external state (e.g., charge) when exposed to a specific sequence of environmental conditions.

In this case, the control problem is differentiated from traditional deterministic control in the face of random uncertainty by the fact that the actuator authority will never be sufficient to drive the system along a specific desired trajectory. Instead, the best that can be hoped for is to influence the transition probabilities with the goal of having the statistics of the system's trajectory be as close as possible to some desired ideal (e.g., maximize the frequency some specific desirable site is visited or minimize the time spent over some undesirable site). Three related problems are addressed:

1. Tailoring the gains to effect a desirable mapping between internal state and recently experienced environment.

In this problem, parameters of a mapping between the environment and an internal state are selected such that the likelihood that the internal state is in a desired condition when the system is in a desired Markov state is maximized and the likelihood that it is in an undesired internal state when the system is in an undesired Markov state is minimized. Here, ‘internal’ means that the state of the system cannot substantively influence the transition probabilities for the Markov chain. As a concrete example, a particle with variable porosity can become porous at a desired site for drug delivery but remain comparatively impermeable elsewhere.

2. Tailoring the gains to effect a mapping between external state and recently experienced environment that brings the long-run or transitions probabilities of the Markov chain closest to desired values.

In this problem, the parameters of a mapping between the environment and an external state are selected such that the likelihood of visiting certain desired Markov states is maximized and the likelihood of visiting undesired ones is minimized or constrained. Here, ‘external’ means that the state of the system can influence the transition probabilities for the Markov chain. As a concrete example, a particle with variable charge or buoyancy can become charged or buoyant at a junction where one branch is to be favored but remain neurally charged or neutrally buoyant elsewhere.

3. Tailoring the gains to effect a mapping between external states and recently experienced environment in the face of energy constraints.

In this problem, we envision the same problem as above, except in the face of a set of energy constraints that limit the set of feasible trajectories. Specifically, we assume that each Markov state has associated with it some random level of energy transfer rate to or from the system, characterized by a mean and deviation, so that any walk through the Markov chain generates some expected net cumulative energy transfer; we then require that the expected cumulative net energy transfer be nonnegative (a sequence that generates a net negative cumulative energy transfer will lead to an untimely stoppage), exceed some threshold, or fit within some bounds.

In all three of these problems, the tailored system is modeled as having a set of observable sensed variables that correlate with the Markov state (a hidden Markov model) and a set of internal and external actuators (e.g., porosity, camera on/off, flight controls) that can be driven by expending energy, with the latter having some influence over the external trajectory of the system and management of the former providing a means of energy conservation (e.g., turn off a sensor when not in use, remain impermeable when not in use, preserving chemical potential).

A number of control approaches can be considered, with each providing some parameterized mapping between the sensed variables and adjustments to the actuators.

The primary controller embodiment of interest is a multilayer perceptron network composed of a bank of thresholding functions that take as their input weighted sums of the sensed variable and that are fed into an AND function (everything must pass); in a related embodiment, they are summed after weighting and fed into another threshold function (some fraction must pass); In some embodiments they are passed into some other such mechanism. In all of these embodiments, the parameters to be optimized are the weights of the sums or, where appropriate, the threshold levels (generally, these would be normalized against the weights). Note that this mechanism is fundamentally a collection of linear classifiers that together are used to select an action, and the optimization problem is one of specifying each classifier.

The specific optimization problem is then structured as choosing the parameters (e.g., the slopes of the linear classifiers) in a way that balances the maximizing of some goal function, for example the fraction of time spent near desired location, while minimizing some penalty function, for example the non-uniformity of the distribution of time spent over undesirable locations, while also satisfying some set of constraints (e.g., expected cumulative net energy consumption remains always below some level).

Standard techniques familiar to those skilled in the state of the art of machine learning and optimization can be used to solve the optimization problem using theory or heuristics in a manner that is optimal or boundedly suboptimal. These can include the development and backtesting of a support vector machine using a subset of gathered experimental data. In the specific instance of designing for a drug delivery particle, blood tests can be taken at a variety of locations and a subset of these used to compute the parameters of the support vector machine, which is then tested against the remaining data.

### Fabrication of Environmentally-Sensitive Particles

In some embodiments, the presently disclosed subject matter relates to the fabrication of environmentally-sensitive materials and particles useful for the purposes of targeted drug delivery, but is also applicable more generally to the design and programming of any tailored system to respond in an appropriate way to changes in environment. In such embodiments, a core idea of the presently disclosed subject matter is to assemble from lipid bilayers, polymers films, etc. a composite material composed of layers of materials, mixtures of material elements, nested or neighboring shells, etc. in such a manner that the assembly exhibits a unique response when subjected to a particular environment and does not exhibit that response when subjected to a different environment, the composition being selected via a mathematical methodology.

Physical Problem. In some embodiments of the presently disclosed subject matter, in any of its various embodiments, a material particle or assembly is generated which exhibits a measurable sensitivity to its environment or to the sequence of environments to which it is subjected. In some embodiments, the objective is to cause a particle to release a carried payload such as a drug when it is in a specific location within the circulatory system of an animal or plant; in a related application, it is to cause some change in conformation or other externally-sensible feature of the particle which has the effect of steering the particle on the basis of environment as it makes its way through the circulatory system.

Mathematical Problem. The specific mathematical problem of interest is for the case of a continuous-transition, discrete-state Markov chain of with a single chain of finite extent and no periodic states; this is ergodic with stable long-run probabilities (if the chain is very large or infinite, it can be divided into a set including the initial state within which there is a high probability or remaining over some arbitrary time horizon, and a set which there is a low probability of entering). Such a chain is representative of the movement of blood and the particles it carries through the circulatory system. At each juncture, there is some probability of taking one branch over the other, but the system is closed so that every juncture can eventually be visited after a long enough period of time. A particle or system can move through this network with the particle or system having some means of causing changes in its internal state (e.g., its porosity, whether a sensing mechanism is on, etc.) or external state (e.g., charge) when exposed to a specific sequence of environmental conditions.

In this case, the control problem is differentiated from traditional deterministic control in the face of random uncertainty by the fact that the actuator authority will never be sufficient to drive the system along a specific desired trajectory. Instead, the best that can be hoped for is to influence the transition probabilities with the goal of having the statistics of the system's trajectory be as close as possible to some desired ideal (e.g., maximize the frequency some specific desirable site is visited or minimize the time spent over some undesirable site).

Physical Compositions. A number of approaches familiar to those skilled in the state-of-the-art of chemistry, biology, and bottom-up and top-down nano- and micro- fabrication to physically generate particles which can be made to be responsive to the environmental experience.

In general, a composite tailored material can be modeled as a network of mass flow elements that is analogous to a network of electronic devices, with porous materials analogized as electrical resistors, contained volumes analogized as capacitors, stores of chemicals analogized as batteries, etc. This understanding of mass transport networks will be familiar to those skilled in the state of the art of mass transport. Like electrical networks, mass transport networks can be connected into circuits that encode certain computational functionalities, and the parameters of the elements of these networks can be selected to exhibit specific types of functionality, a process that is analogous to coding in hardware.

For the specific problem of interest, it is desirable to realize through a simple fabrication process a material that releases a dose of a drug when subjected to a specific environment or sequence of environmental conditions that would be representative of travel to a particular region of the body, but which does not release the drug when subjected to other sequences, which are representative of travel to other regions of the body.

In some embodiments, a particle is formed as a composite shell composed of alternate layers of material that is resistive to the flow of a solute and of a material that is stores the solute, with this shell enveloping a volume that is high is concentration of the drug. The first material represents a resistance, the second a capacitance, and the enclosed volume a battery or large charged capacitor. The enveloped volume might be a volume of liquid with a high concentration of drug contained within it, a soluble particle or droplet of drug, a particle of soluble or permeable solid impregnated with the drug, etc. The capacitive volume might be a thin film of water or some other solvent; a sponge-like layer capable of absorbing water or some other solvent, like that used for contact lenses; or some other material that can serve as a capacitive volume. The resistive material can be a lipid bilayer like those used to form liposomes, a porous polymer, a thin film of neighboring or overlapping solid chips, or some other such material.

By using different types of resistive material, each of which exhibits a more or less porosity, depending on the environment to which it is exposed, the composite shell can be made to function in such a way that it is highly likely to releases at a target location and is highly unlikely to release at other locations.

The operation of a particle of this form is as follows: when the particle is not in the region of interest, but is moving along a trajectory through the circulatory system that will lead it to do so, layers from the innermost to the outermost will become porous sequentially such that the capacitive layer between each is able to charge. So, for example, the first resistive layer will become porous when exposed to an environment like that along the first leg of travel to the target site, allowing the first capacitive layer to charge up to the concentration within the core volume. Then, when the next leg is reached, the next resistive layer will become porous, and the second capacitive layer will charge. This process continues until the particle is at the delivery site, at which point the outermost layer becomes porous, allowing the dose of drug stored within the various capacitive layers to discharge out of the particle and into the environment. After the particle leaves the target region, the layers become impermeable and the process starts again. If the particle takes a track that does not lead to the desired target area, the number and sequence of layer openings will not be correct and it will not be possible to achieve a release except in rare circumstances, these forming false positive events in a statistical sense.

A number of variants of this embodiment can be implemented, all varying slightly in their method of operation, but being substantially similar to that describe above. In some embodiments, the innermost layer is closed when the outermost is open, causing only the dose within the capacitive layers to be released; in another, all layers are open at the time of release so that a dose flows from the central volume to the surroundings. In some embodiments, when on the path leading to the target site, the layers open sequentially and remain open in all environments downstream of the one first opening, causing each capacitive layer to charge to the same concentration as the central source volume; in another, only one resistive layer is open at a time, causing the charge within the capacitive layers to cascade from one capacitive layer to the next, dropping by roughly half with each cascade; in others, layers open out of sequence but remain open so that when the target area is reached, the source is connected to the target environment through a high-porosity pathway; in others, some combination of these happens.

In some embodiments, the source volume of solute is located in a central volume; in others, it is one or more layers interleaved with the capacitive and resistive layers. In others embodiments, small spherical source volumes are each enveloped by one or more resistive layers and distributed throughout a capacitive volume that is itself bounded by resistive layers; in some of these the encapsulated source particles are contained within a larger spherical volume; in others, they are contained in a thin layer between two spherical shells or planar or cylindrical layers.

In some embodiments, the source of drug can be distributed throughout the various capacitive layers so that these charge individually and continuously, but only become interconnected when a specific environmental sequence is experienced. In this embodiment, which has the advantage of being easy to fabricate, capacitive layers only charge up substantially when they've had a long period of being sealed on each side by impermeable resistive layers; this occurs when they are on tracks that are not those leading to the target location. When the particle is on a track towards release, the capacitive layers communicate with each other and eventually release as a group.

In some embodiments, release does not depend on the sequence of environments being experienced, but only on a very specific environment being experienced. Some of these embodiments can involve a volume encapsulated by a single resistive film or multiple layers of film like those already described, but all of the same type. Others can involve a series of capacitive layers separated by resistive layers, all of the same type. In these embodiments, the resistive film is more porous in environments like those found in the vicinity of the target area and less porous in other environments. This differential of porosity leads to the drug or other solute being released preferentially at the target location.

The fabrication of all of these embodiment and others can be achieved by way of a number of different fabrication techniques. In some embodiments, lipid bilayers like those used in liposomes or synthetic biology are used; in others, polymer films or particles are used; in others, other techniques are used.

In some embodiments, fabrication is achieved by structuring the particle as a multi-layer liposome carrying a water-soluble drug, with each resistive layer formed by a variable-porosity lipid bilayer and each capacitive layer formed by a thin film of water. In some embodiments, such liposomes are formed by a process of lipid bilayer formation and extrusion to a given diameter, which correlates with number of layers. In some embodiments, if different formulations are used for each bilayer, the processes of encapsulation and extrusion can be staged in sequence to give a multi-layer liposome with the desired mix of layers. In some embodiments, also with different formulations for each layer, the different films are formed at once and their sequence of ordering from inner layer to outer layer is random for each individual liposome.

In some embodiments, a lipid bilayer serves as capacitive layer and a water film serves as the resistive layer; this variation is relevant when a drug is insoluble in water but can be dissolved in an oil or the lipid bilayer. In some embodiments, the lipid bilayers can form up as spherically-symmetric spherical shells; in others, as cylindrical rolls; in others, as sheets; in others as some other shape. Formation and sorting of these various shapes will be familiar to those skilled in the state of the art.

In some embodiments, the particle is formed from polymers by way of a top-down process. In some embodiments, a film of one type of polymer is deposited as a capacitive layer and a film of another type of polymer is deposited on top of this film as a resistive layer. The resulting multi-layer film is then rolled up as a cylindrical roll.

In some embodiments, film is rolled around a cylindrical core that is composed of or impregnated with the drug; in embodiments, the cylindrical core is a polymer impregnated with the drug or a metal coated with or impregnated with it. These embodiments are similar in nature to drug-eluting stents, which have a high-concentration of drug separated from the environment by a polymer through which the drug diffuses, except that in the embodiments described here, the resistance of the barrier material is a function of environment so that elution only occurs in the presence of a specific environment or sequence of environments.

In some embodiments, which form an alternate application to the preferred application described here, a drug-eluting stent or similar biomedical device is coated with a film made of a polymer or other suitable material, which film is sensitive to environment. The sensitivity to environment of the film is selected in such a way that the stent or other device only elutes when certain environmental conditions are present in the region of the the blood stream where the stent is located. If other conditions exist, these film will be relatively more impermeable. In this way, the release of the stent can be shut off if the person in which it is placed is subject to some experience which would make the release of the drug dangerous. As a concrete example, if there would be a negative interaction with the drug and other drugs that can be in the blood stream, elution will be turned off if those other drugs are detected by the film. Or, as another concrete example, a stent that can elute a blood thinner does so when chemical or environmental indicators suggestive of an impending blockage are detected, but otherwise does not; or, normally does so, but stops when indicators suggest that continued release would be dangerous.

In some embodiments, the capacitive layer itself is impregnated with particles of drug and the roll has no core or some inert core. As a specific example, a polymer with a high water content, such as those used in contact lenses, can serve as the capacitive layer and can be impregnated with particles that elute a drug into it. This polymer layer can be capped by resistive films of variable porosity which regulate release of the drug that accumulates in the capacitive layer from it into the environment. The two- or three-layer film can then be rolled up as a cylinder and cut into short segments giving short cylindrical particles as the drug-delivery particle.

In some embodiments, polymer films are formed by way of a process known as spin-coating. In this process, a solid substrate like a silicon wafer is spun and the polymer in an uncured form, or its precursor, is poured onto the spinning wafer, forming a thin film. The thin film is then cured into a solid or rubber-like elastic solid via some process like exposure to UV radiation, heat, chemicals, etc. A second layer is built upon the first in the same manner. In some embodiments, each one or more of the films has embedded within it chemical or particulate additives. In some embodiments, these are added in bulk before curing; in others, they are added by a process of patterning and impregnation after the formation of the film but before curing; and in still others they are added after curing. In some embodiments, after all of the layers are complete, the multi-layer sheet of film is cut into small pieces which are allowed to self-roll; in others, the sheet is rolled into a long thread-like cylinder and then cut into short pieces; in others, some other technique is used to form the sheet into individual particles.

These various fabrication techniques will be familiar to those skilled in the state of the art. The techniques for forming and rolling the spin-coated films have been demonstrated by researchers at the Massachusetts Institute of Technology in the context of fabricating polymer bands which change color under strain.

In the polymer-based embodiments described above, the form of the particle is a cylinder, but In some embodiments formed using similar materials or by way of similar processes, the shapes can take the form of spherical particles, sheets, etc., depending on the details of the processing technique used.

In some embodiments, which are variations on the former, a single film composed of a matrix and an additive is used, with one serving as the capacitive medium and the other serving as the resistive medium. The additive can be a powder of particles or fibers, or a connected mesh or truss-structure, or some other such material. The matrix can be a solid, a gel, or some other such material. Depending on the environment, the conformation of the particle can change is some desirable way. For example, in some embodiments, a particle composed of a mesh of environmentally-sensitive fibers can be embedded within a compliant polymer can contract when environmental conditions cause the mesh fibers to shorten, closing up the porous spaces between the nodes of the mesh and limiting diffusion out of the particle and also changing its size. In some embodiments, source particles can be distributed throughout a sponge-like matrix enveloped by a stiff film and environmentally-driven expansion or contraction of the matrix can change its resistance to movement of solute through it.

In some of the above embodiments and in some other embodiments, the films are composed of locally distinct regions, each with their own features such as level of sensitivity to various different environmental signatures. In some embodiments, the patterning is accomplished by a top-down process of masking the polymer, applying a paste of the dopant or submerging the masked film and substrate into a solution containing the dopant and allowing it to diffuse into the polymer. In another , different sources of dopant are printed onto the film via ink-jet printing, screen-printing, or some similar method that deposits droplets or coats of this material; the dopant is allowed to leach from the coating into the polymer; and the coating is then stripped. These various top-down approaches are similar in general concept to doping a solid film in, for example, semiconductor fabrication. In another class of embodiments, the film is itself composed of a number of subparticles that have been assembled and fused or bonded by way of some intermediate matrix or bonding material; this approach is sometimes seen in large-scale rubber mats that are from chopped and fused multi-colored rubber pieces. In yet another class of embodiments, the films are formed by depositing droplets of different liquid polymer precursors or mixes, allowing these droplets to spread up against each other as the liquid levels, and then curing the polymer. In still other embodiments, the film starts as a variety of beads that are mixed and spread and then pressed into a film using heat and pressure or some other such mechanism; at a larger scale, this approach is found on some arts and crafts techniques.

Regardless of the embodiment, the key feature is that the film is formed of neighboring regions that each have different responses to their environment so that the overall porosity of the film will be a scalar function of some vector of environmental variables or sequence of environmental variables.

In some embodiments, the presently disclosed methods relate to formulating and fabricating environmentally-sensitive drug particles which exhibit location-specific release within the circulatory system after the fashion of terrain contour matching. In some embodiments, the presently disclosed subject matter addresses targeted drug delivery using micro- or nano-fabricated particles that estimate of their own location within the body and release drugs near target locations selected on the basis of offline medical imaging.

Targeted delivery techniques commonly use chemical targeting via cell-borne receptors, genetics, or an ex vivo stimulus such as heat or radio waves that prompts spatially-localized release.

With respect to the presently disclosed subject matter, particles or formulations that estimate their own location within the body by correlating vectors of sensed environmental variables (e.g., temp., pressure, salinity, sugar levels, pH, etc.) against a carried map release their drug in the vicinity of a target site on the basis of this location estimate; this approach closely related to terrain contour matching (TERCOM), used in aircraft navigation. In some embodiments, top-down particle formulations are realized: thin sheets of permeable hydrophilic gel, clad with a quilt of polymer materials and charged with an eluting drug, are fabricated by spin-coating and dicing, then allowed to form swiss rolls; in another embodiments, multilaminar shells of environmentally-sensitive lipid bilayer, separated by thin films of water and enveloping droplets of drug in solution, are fabricated chemically. In both such embodiments, the barrier layers are quilted irregularly from a variety of semipermeable materials, each sensitive to a different stimulus, the path length for diffusion to the surroundings (the ‘Manhattan distance’) is a scalar function of the environmental vector in a manner approaching a perceptron.

In designing the detailed composition of such materials, the circulatory system is modeled. In some embodiments, the circulatory system is modeled as a parameterized, closed, lumped-element flow network of resistors, capacitors, and fins, driven by an actuator disc, and embedded within a field, itself modeled as a coarse unstructured mesh of heat-generating and reactive malleable solid, with which it locally exchanges heat and solutes, giving rise to predictable variations in the local circulatory environment. Its traverse is modeled as a discrete-state, continuous-transition Markov process with region of the body (mesh element) as Markov state. The Markov process is cast as a source of symbol sequences representing the path taken during a circuit through the circulatory network; the lumped-element model is cast as a transducer which maps place to environment.

This modeling approach requires the estimation of model parameters. In some embodiments, parameters of the model can be estimated from synthetic tissue analogs used for surgical device development, and particle design (specification of the pattern and composition of the particle layers) is cast as a problem of robust optimization of material parameters with a goal of balancing type I and II errors in release.

The particular sequence of environments that a particle experiences during a circuit of the circulatory system can depend on the context; that is, it can depend on whether the individual is sitting, standing, recline, etc. and on whether they are at high altitude, low altitude, in a warm environment, in a cool environment, mostly indoors, mostly outdoors, etc. If a particle or formulation is engineered only release under certain contexts (in addition to at a specific location), then the release will be targeted to location or context, or both.

By way of example and not limitation, the polymer coating of a drug eluting stent can be engineered to limit the rate of elution when certain environmental conditions are experienced by it (or when not). Because the stent does not move, the shifts in its environment will be related to shifts in context rather than location, so the environmental selectivity of the release mechanism will have the effect of being be selective for context rather than location.

In some embodiments, such as those where a particle is traversing the circulating system, the particle or formulation can be engineered to release only when in the desired location and under the desired context. Or, in some of these other embodiments, the context can be used to prevent release except under certain conditions.

Besides being useful for enhancing the effectiveness of a therapeutic, such a mechanism can allow the implementation of a digital rights management mechanism for engineered therapies, be they genetic or chemical. For example, in some embodiments, an engineered or tailored material is used to limit release so that is only occurs under authorized conditions; in others, an engineered organism or synthetic biological system can perform similarly.

In still other embodiments, release can be structured to occur only when both a certain context and a certain key are present, be that release specific to location as in targeted-release or fixed in the circulatory system as with a stent. In some such embodiments, the key can take to form of another chemical or marker being present; in some of these, a specific RNA strand can serve as key, for example. In all of these embodiments, and in other similar embodiments, the tailored material or mechanism can be said to be implementing drug delivery under a permissive action link.

### Conformation-changing Peptides and Polymers

In some embodiments, the presently disclosed subject matter relates to targeted drug delivery using synthesized peptides or other synthetic polymers or particles that evolve their conformation in response to the local environment found at their their location within the body, or on the basis of their being exposed to a sequence of such environments, in such a manner that the release of a carried drug by the particle or the therapeutic activity or chemical activity of the particle is made a function of location within the body or circulatory system.

Targeted delivery techniques commonly use chemical targeting via cell-borne receptors, genetics, or an ex vivo stimulus such as heat or radio waves that prompts spatially-localized release. As set forth herein, in some embodiments particles or formulations release their drug in the vicinity of a target site by estimating their own location and releasing when in the target vicinity. These techniques are analogous to terrain contour matching (TERCOM) and digital scene matching area correlation (DSMAC), techniques used in aircraft navigation.

Here, a particle changes its conformation in response to environment, and the chemical activity or therapeutic activity can be made to be a function of the particle's location within the body. As with the approaches described in the referenced applications, the particle effectively estimates its location by way of correlation with measured environmental signatures and then links its activity to this estimate or to sequences of the same.

In some embodiments, a large molecule drug with this engineered sensitivity is designed to be inert when not is the physical vicinity of a target location and active when in that vicinity. In some embodiments, the particle serves as a ‘sabot’ for a particle of drug, releasing itself from the active particle when in the proper location. In this second instance, the delivery functionality is separated from the therapeutic functionality.

It is noted that in any of the embodiments of the compositions and methods of the presently disclosed subject matter, the particles, delivery vehicles, and/or formulations need not alter their compositions and/or conformations in response to variations in vivo environmental stimuli in a binary manner. Thus, when the present disclosure refers to a change in composition and/or conformation that results in one or more desired activities, that desired activity need not be entirely absent when the compositions and/or conformations are in the “inactive” form. Rather, contemplated within the scope of the presently disclosed subject matter are compositions and/or conformations in response to variations in vivo environmental stimuli that can be matters of degree. By way of example and not limitation, an “active” composition or conformation can be one that releases detectably more of a drug and/or that has some quantitatively or qualitative increased level of activity although the composition or conformation could have some detectable level of activity when in the “inactive” form provided that whatever difference exists between the active and inactive forms can be measured and/or has some biological and/or therapeutic relevance.

Different embodiments can make use of different combinations of environmental variables. In some embodiments, particles are sensitive to one or more of temperature, pressure, salinity, sugar levels, pH, etc., or are sensitive to one or more other environmental variables, or to combinations involving one more of all of these. The particles can be considered to be estimating their own location within the body by correlating vectors of these sensed environmental variables with carried map represented by the design parameters of the particle, making this approach similar to terrain contour matching (TERCOM) or other similar techniques such as digital scene matching area correlation (DSMAC).

In some embodiments, the particles are realized in a bottom-up fashion. In some embodiments, they are realized via chemistry or multistep combinations of chemistry and processing. In some embodiments, they are realized via a process of polymer synthesis. In some embodiments, the polymers are long chains. In some embodiments, they are realized via a process of peptide synthesis. In some embodiments, they are realized via a process of protein synthesis. In some embodiments, they are realized via synthesis of a protein complex. In some embodiments, they are realized via some other bottom-up chemical or nanofabrication process.

In some embodiments, the particles are realized in a top-down fashion. In some embodiments, they are realized by implementing chips using top-down micro- or nanofabrication processes; in some embodiments, these chips are biodegradable or inert; in some, they carry a releasable payload. In some embodiments, they are realized by top-down fabrication of meta-particles formed from long strands that fold upon each other in one or more ways, depending on environment, and, therefore, behave like a protein. In some embodiments, a hair-sized or smaller polymer strand is formed with regions doped or coated or otherwise treated in such a manner as to cause the strand to fold in a particular fashion. In some embodiments, the particle is a folded strand of memory material; in some, this memory material is a memory metal; in some, it is a memory metal coated with a polymer coating; in some, it elutes a drug in some folded conformations but not others, or its rate of elution is a function of its specific conformation; in some, it is a drug which elutes, and its rate of elution is a function of its specific conformation.

In some embodiments, the particles are designed by selecting the parameters of their design such that the particle conformation exhibits the desired behavior as a function of the environment of the circulatory system at different locations. In some embodiments, in designing the particles, the circulatory system is modeled. In some embodiments, the circulatory system is modeled as a parameterized, closed, lumped-element flow network of resistors, capacitors, and fins, driven by an actuator disc, and embedded within a field, itself modeled as a coarse unstructured mesh of heat-generating and reactive malleable solid, with which it locally exchanges heat and solutes, giving rise to predictable variations in the local circulatory environment. In some embodiments, the traverse of the body or circulatory system or other system is modeled as a discrete-state, continuous-transition Markov process with region of the body (mesh element) as Markov state. The Markov process is cast as a source of symbol sequences representing the path taken during a circuit through the circulatory network; the lumped-element model is cast as a transducer which maps place to environment.

This modeling approach requires the estimation of model parameters. In some embodiments, parameters of the model are estimated from synthetic tissue analogs used for surgical device development, and particle design (specification of the pattern and composition of the particle layers) is cast as a problem of robust optimization of material parameters with a goal of balancing type I and II errors in release or activity.

The particular sequence of environments that a particle experiences during a circuit of the circulatory system can depend on the context; that is, it can depend on whether the individual is sitting, standing, recline, etc. and on whether they are at high altitude, low altitude, in a warm environment, in a cool environment, mostly indoors, mostly outdoors, etc. If a particle or formulation is engineered to only release or become active under certain contexts (in addition to at a specific location), then the release will be targeted to context, or to both location and context.

In some embodiments, a drug in the form of a synthesized peptide can have its sequence of amino acids selected such that the potential energy well of the peptide, which varies with conformation, exhibits several local minimums, one of which can be a global minimum, each separated by a barrier with its own activation energy, measured from the base of the minimum to the peak of the area. Through proper amino acid sequence selection, the relative levels of these minima and the levels of the activation energies separating them can be made to be some function of the environment of the circulatory system, with such factors as temperature, salinity, oxygen level, lighting level, etc. playing a role. If properly selected, the barriers will change over time in such a way that convergence of the conformation to a particular state will be preferred under one environment and convergence to a different state will be preferred under a different environment, the rate of convergence depending in the heights of the energy barriers. For example, the sequence can be engineered to cause the particle to take on an inert conformation in certain environments where activity of the particle is not desired and to take on active conformations in environments where the particle would be therapeutic. In some embodiments, the progression of changes in the shape of the potential energy well can be structured such that a particle can achieve a specific desired configuration, such as a therapeutic one, only if a specific sequence of environments is experienced. In some embodiments, this is accomplished by structuring changes in the heights of the activation energy barriers such that a particle must move to a second conformation from a first before it can move to a third and this can only happen along a specific path. Along other paths, the barrier between the first and second and the first and third remained large so that transition to the third cannot occur even when the barrier between the second and third is lowered, the second never having been reached. In some embodiments, other, similar, such arrangements involving more than one local minimum are used.

In some embodiments, the changes in environment experienced by the particle result from changes in environment caused by changes in context rather than by those caused by location, or by changes caused by the combination of location and context. In these embodiments, the environment can change due to, for example, an increased rate of respiration, an increased metabolism, sleeping, or some other such change in context. In some embodiments, the particle is sensitive only to changes caused by changes in location of the particle within the body.

For example, in some embodiments, the conformation of a particle at some fixed location such as at a stent is engineered to cause it to be inert and attached when certain environmental conditions are experienced but active and free-floating when others are experienced. Because the particle does not move until active, the shifts in its environment will be related to shifts in context rather than location, so the environmental selectivity of the release mechanism will have the effect of being be selective for context rather than location.

In some embodiments, such as those where a particle is traversing the circulating system, the particle or formulation can be engineered to become therapeutically active or release only when in the desired location and under the desired context. Or, in some of these other embodiments, the context can be used to prevent release or activity except under certain conditions.

Besides being useful for enhancing the effectiveness of a therapeutic, such a mechanism can allow the implementation of a digital rights management mechanism for engineered therapies, be they genetic or chemical. For example, in some embodiments, a large molecule or some other tailored particle is designed for activity or release only under select conditions such that release or activity only occurs under authorized conditions. An engineered organism or synthetic biological system can perform similarly, either through genetic mechanisms or by incorporation of the aforementioned embodiments.

In still other embodiments, activity or release is structured to occur only when both a certain context and a certain key are present, be that activity or release specific to location as in targeted-release or fixed in the circulatory system as with a stent. In some such embodiments, the key can take to form of another chemical or marker being present; in some of these, a specific RNA strand can serve as key, for example; in others, a specific molecule such as an antibody can serve as key.

In embodiments just described, where activity or release is made sensitive to context, in whole or in part, and in other similar embodiments, the tailored molecule or mechanism can be said to be implementing drug activity or drug delivery under a permissive action link.

An advantage of this mechanism is that the drug or drug delivery mechanism can be tailored to activate or release in a location within the body circulatory system without requiring specific knowledge of the disease being addressed or the cells to which the therapy is being applied and also without using external targeting aids such as heat pads or radio waves.

Additionally, particles of the sort described can be used to introduce location-sensitivity to other problems in mass transfer and chemistry or can be used for more complex targeting problems such as context-sensitive drug targeting or release.

### REFERENCES

All references cited in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or compositions employed herein.

Hsiao et al. (2017) Automated Modeling of Large-Scale Arterial Systems. in MIT Microsystems Technology Lab (MTL) Annual Research Report, page 21.

### Lassoued et al. (2017) A Hidden Markov Model for Route and Destination Prediction. 2017 IEEE 20th International Conference on Intelligent Transportation Systems (ITSC). DOI 10.1109/ITSC.2017.8317888.

Tanner et al. (2010) Experimental demonstration of lossy recording of information into DNA. in Proc. SPIE 7679, Micro- and Nanotechnology, Sensors, Systems, and Applications II, 77920; doi:10.1117/12.858775

### U.S. Patent Application Publication No. 2009/0275031.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for targeted drug delivery via a mechanism that uses a particle's internal estimate of its own location within a subject's body to target release of a drug contained therein and/or thereon at a point specified by offline medical imaging.

2. The method of claim 1, wherein the particle's estimate is formed in part on the basis of information it detects and records about the environment of its recent past.

3. The method of claim 1, wherein delivery is accomplished by tailoring the particle's composition so that it releases the drug when exposed to a specific sequence of environmental conditions or some set of specific sequences of environmental conditions, but does not do so when exposed to the specific sequence of environmental conditions or the set of specific sequences of environmental conditions.

4. The method of claim 3, wherein the specific sequence of environmental conditions corresponds to the particle being at a specific location, such as at a unique cluster of capillaries, within the body of an animal or the vascular system of a plant.

5. The method of claim 1, wherein the particle comprises a synthetic biological organism or mechanism and delivery is accomplished by tailoring the synthetic biological organism or mechanism so that it expresses a specific gene or releases a drug or a chemical when exposed to a specific sequence of environmental conditions or some set of specific sequences of environmental conditions, but does not do so when exposed to the specific sequence of environmental conditions or the set of specific sequences of environmental conditions.

6. The method of claim 5, wherein the particle's estimate is formed in part on the basis of information it detects and records about the environment of its recent past.

7. The method of claim 5, wherein the specific sequence of environmental conditions corresponds to the particle being at a specific location, such as at a unique cluster of capillaries, within the body of an animal or the vascular system of a plant.

8. The method of claim 1, wherein passage of a tailored material or synthetic biological organism or system through some network of vessels can be treated as a brute force decryption, with the targeted capillary as message, the location-sensitive particle as cryptogram, the trailing history of branch environments as trial key, circulation as a random cycling of trial keys, release as a successful decryption, and particle design as a problem of robust optimization of material parameters with a goal of balancing type I and II errors in release.

9. The method of claim 1, wherein the particle comprises an endowment of usable energy or a mechanism for energy harvesting and storage, and further wherein the particle is constructed to use the endowed or harvested and stored energy to influence its movement through the circulatory system in such a way as to increase the frequency or likelihood of it visiting a desired target location.

10. A method for drug delivery, wherein the probability of a circulating drug particle taking one branch over another at one or more junctions in a subject's circulatory system is controlled by a mechanism that couples changes in some expressed feature or collection of expressed features of the particle that affects its interaction with its environment to the environmental conditions at or leading up to the branch in such a manner as to increase the likelihood of the particle visiting or revisiting a particular targeted area through its course of circulation.

11. The method claim 10, wherein the expressed feature of the particle is its buoyancy.

12. The method of claim 10, wherein the expressed feature of the particle is its coefficient of drag.

13. The method of claim 10, wherein the expressed feature of the particle is its electric charge.

14. The method of claim 10, wherein the expressed feature or collection of expressed features of the particle is a combination of its buoyancy and its coefficient of drag.

15. The method of claim 10, wherein the mechanism which couples the environmental conditions to changes in the expressed feature is a tailored material.

16. The method of claim 15, wherein the mechanism that couples the environmental conditions to changes in the expressed feature is a tailored composite material.

17. The method of claim 16, wherein the tailored composite material is designed by solving an optimization problem that specifies the composition of the material which maximizes time spent at the target site while minimizing or constraining the amount of time spent at any other particular site.

18. The method of claim 16, wherein the tailored composite material is designed by solving an optimization problem that specifies the composition of the material which maximizes its rate of delivery to a target site while minimizing or constraining its rate of delivery to any or all non-target sites.

19. The method of claim 10, wherein the mechanism that couples the environmental conditions to changes in the expressed feature is a synthetic biological mechanism.

20. The method of claim 19, wherein the mechanism that couples the environmental conditions to changes in the expressed feature involves a recording in DNA of the recent history of environmental conditions experienced by the particle or other delivery vehicle.

21. The method of claim 20, wherein the synthetic biological system is designed by solving an optimization problem that maximizes time spent at the target site and/or a release rate in the vicinity of the target site while minimizing or constraining the amount of time spent at any other particular site or the release rate at any other pre-determined site.

22. The method of claim 10, wherein permeability, porosity, or some other internal feature of the particle that controls the rate of release of the drug is regulated and design of the particle for steering and release is coupled.

23. The method of claim 22, wherein a regulatory mechanism is designed by solving simultaneous optimization problems for both steering and release in such a manner that release rate is maximized in the vicinity of the target site and minimized or constrained at any other pre-determined site.

24. The method of claim 10, wherein a formulation comprising several components is employed rather than a singular particle, and the formulation collectively implements a regulation mechanism.

25. The method of claim 24, wherein the formulation comprises a steered particle that carries and releases a drug and is sensitive to concentrations of a plurality of markers selected from the group consisting of a release marker, a steering marker, a particle that carries and releases a steering marker, and a particle that releases a release marker.

26. The method of claim 25, wherein the formulation comprises a steered particle that carries and releases a drug and is sensitive to the concentration of two markers, a release marker and a steering marker, a particle that carries and releases both of these markers.

27. A method for fabricating an environmentally-sensitive particle comprising cladding a hydrogel or other hydrophilic medium with a comparatively impermeable layer, the latter of which has a permeability that is sensitive to environment, and thereafter arranging layers of such materials upon one another.

28. A method for fabricating an impermeable film that is selective for its environment comprising quilting together a plurality of tiles or bits of different materials, each with its own response to an environment to which the impermeable film might be exposed.

29. A method for fabricating an impermeable film that is selective for its environment comprising depositing randomly in a lipid bilayer or other similar film a plurality of compounds that change at least one characteristic in response to different environmental stimuli.

30. A method for fabricating a material the permeability of which is sensitive to environmental stimuli comprising arranging layers of comparatively permeable material separated by layers of semipermeable material in such a way that the total path traveled by a diffusing particle depends on the distance separating pores in the semipermeable material, wherein the distance separating pores in the semipermeable material varies with different environmental stimuli.

31. Use of physical parameters, optionally material parameters, and geometry from a synthetic tissue model or whole-body synthetic tissue model to specify design parameters of a selective-release drug delivery mechanism.

32. Use of an environmentally-sensitive material, particle, or formulation, to subject targeted release of a drug to a form of permissive action link.

33. A method for fabricating an environmentally-sensitive large pseudomolecule comprising extruding, patterning, or otherwise processing a strand of polymer that is locally doped, coated, or otherwise treated, thereby causing the strand to fold into a conformation that is sensitive in a pre-determined way to environmental stimuli.

34. The method of claim 33, wherein the environmentally-sensitive large pseudomolecule is sensitive to environmental stimuli in a manner that results in a conformation of the environmentally-sensitive large pseudomolecule to vary different locations of a subject's a circulatory system.

35. A method for fabricating an environmentally-sensitive large molecule comprising synthesizing the environmentally-sensitive large pseudomolecule from a sequence of monomers, the sequence of which results in the environmentally-sensitive large pseudomolecule to adopt different conformations in response to local environmental stimuli or to a particular sequence of local environmental stimuli in a pre-determined manner.

36. The method of claim 35, wherein the environmentally-sensitive large pseudomolecule comprises a peptide, a protein, a protein complex, or a combination thereof, optionally wherein the monomers are amino acids.

37. A method for fabricating an environmentally-sensitive large molecule comprising synthesizing the environmentally-sensitive large pseudomolecule from a sequence of monomers, the sequence of which results in the environmentally-sensitive large pseudomolecule adopting different conformations in response to different environmental stimuli at one or more locations within a subject's body, wherein at least one of the different conformations results in the environmentally-sensitive large pseudomolecule being therapeutically active and at least one of the different conformations results in the environmentally-sensitive large pseudomolecule being therapeutically inactive.

38. The method of claim 37, wherein the environmentally-sensitive large pseudomolecule comprises a peptide, a protein, a protein complex, or a combination thereof, and optionally wherein the monomers are amino acids.

39. Use of physical parameters, optionally material parameters, and geometry from a synthetic tissue model or whole-body synthetic tissue model to specify the design parameters of large molecule drug or large molecule drug delivery composition, wherein the design parameters result in the large molecule drug or large molecule drug delivery composition selectively expressing its therapeutic activity or selectively concealing its therapeutic activity.

40. Use of an environmentally-sensitive large molecule, tailored material, particle, and/or other formulation to subject activity of a drug associated therewith to a permissive action link.

41. Use of an environmentally-sensitive large molecule, material, particle, and/or other formulation to subject release of a drug associated therewith to a permissive action link.

42. Use of an environmentally-sensitive large molecule, tailored material, particle, and/or other formulation to subject an activity of a drug associated therewith to targeting dependent on its location within a subject's body.

43. Use of an environmentally-sensitive large molecule, tailored material, particle, and/or other formulation to subject release of a drug associated therewith to targeting dependent on its location within a subject's body.

44. A method for synthesizing a composition, the conformation of which is sensitive to its location within a subject's circulatory system, comprising selecting a sequence of monomers or another feature of the composition, such that the potential energy well of the composition comprises local minima and activation energy barriers that vary with environmental stimuli in such a way as to cause the composition to adopt a desired conformation at one or more pre-determined locations with a subject's body and one or more different pre-determined conformation at other locations within the subject's body.

45. The method of claim 44, where the composition comprises a peptide, a protein, a protein complex, a polymer strand, or any combination thereof

46. The method of claim 44, where the composition comprises or is otherwise associated with a drug.

47. The method of claim 44, wherein sensitivity of a shape of the potential energy well to the environmental stimuli is such that the composition adopts a specific conformation with high probability only when the particle traverses a pre-determined specific path or path segment within the subject's body.

48. The method of claim 44, wherein the potential energy well comprises there three local minima, as a function of conformation, and three activation energy barriers separating them, with the barrier between the first and third always lowered for regions of a flow field on the return circuit, optionally the subject's veins, after a target location or non-target location, optionally the subject's capillaries, has been passed, with the first minimum the global minimum during that time, and with the barrier raised at all other times;
where the barrier between the first and second is only lowered on branches leading to the target area, but not on branches that do not lead to the target area, so that the conformation can only change to the second conformation if the particle flows along a branch leading to the target area; and where the barrier between the second and third conformations is only lowered in the vicinity of a target or non-target (e.g., capillaries), but not during branches leading to or away from these areas, and with the barrier between the first and second kept raised, so that the third conformation can only be reached if the particle takes the correct path, which left it in the second conformation, it being left in the first conformation, even with the barrier between the second and third lowered, otherwise, due to the barrier between the first and second minima.
